(19) United States
(12) Patent Application Publication
(10) Pub. No.: US 2008/0120733 A1
(43) Pub. Date: May 22, 2008

(54) Rnai-Based Method for Selecting Transfected Eukaryotic Cells

(75) Inventors: Martin Hafner, Wolfenbuttel (DE); Karina Nawrath, Braunschweig (DE); Werner Muller, Braunschweig (DE)

Correspondence Address: FOLEY AND LARDNER LLP;SUITE 500, 3000 K STREET NW, WASHINGTON, DC, 20007, US

(73) Assignee: GBF-GESELLSCHAFT FUR BIOTECHNOLOGISCHE FORSCHUNG, Braunschweig (DE)

(21) Appl. No.: 11/629,709

(22) PCT Filed: Jun. 16, 2005
(86) PCT No.: PCT/EP05/06488

§ 371 Date: Dec. 10, 2007

(30) Foreign Application Priority Data
Jun. 16, 2004 (EP) 04014124.4

(51) Int. Cl. A01K 67/027; C12N 15/87; C12N 5/02; C12N 5/00; C12N 5/04; A01H 1/00; C12N 15/00

(52) U.S. Cl. 800/14; 435/455; 435/419; 800/298; 435/325; 435/354; 435/353; 435/366; 435/363; 800/24; 800/278

(57) ABSTRACT

The invention relates to a method for the production of a eukaryotic cell selectable by inactivation or reduction of an endogenous gene function, comprising the steps of (a) introduction of one or more vectors into the cell and (b) expression of a siRNA and preferably shRNA coded by the one or more vectors, directed against an endogenous selectable gene and inactivating same, said siRNA or shRNA being the transcription product of an RNAi selection cassette, the selection cassette comprising a section of at least 19 nucleotides of the transcribed region of the gene, said selection being operatively linked to a promoter and a transcription termination signal. Furthermore, the invention relates to a eukaryotic cell comprising an RNAi selection cassette which is directed against an endogenous selectable gene and inactivates the function of said gene; wherein the RNAi selection cassette comprises a section of the gene with a length of at least 19 nucleotides which is operatively linked to a promoter and a transcription termination signal. Moreover, the invention relates to methods for the production of a transgenic mammal, comprising the steps: (a) injection of the embryonic stem cell according to the invention or of an embryonic stem cell selected according to the method of the invention in blastocysts of a mammal, (b) transfer of the blastocysts into the uterus of a mammal, and (c) carrying the transgenic mammal to full term. Finally, the invention relates to a method for the production of a transgenic plant.

The invention relates to a method for the production of a eukaryotic cell selectable by inactivation or reduction of an endogenous gene function, comprising the steps of (a) introduction of one or more vectors into the cell and (b) expression of a siRNA and preferably shRNA encoded by the one or more vectors, directed against an endogenous selectable gene and inactivating same, said siRNA or shRNA being the transcription product of an RNAi selection cassette, the selection cassette comprising a section of at least 19 nucleotides of the transcribed region of the gene, said section being operatively linked to a promoter and a transcription termination signal. Furthermore, the invention relates to a eukaryotic cell comprising an RNAi selection cassette which is directed against an endogenous selectable gene and inactivates the function of said gene; wherein the RNAi selection cassette comprises a section of the gene with a length of at least 19 nucleotides which is operatively linked to a promoter and a transcription termination signal. Moreover, the invention relates to methods for the production of a transgenic mammal, comprising the steps: (a) injection of the embryonic stem cell according to the invention or of an embryonic stem cell selected according to the method of the invention in blastocysts of a mammal, (b) transfer of the blastocysts into the uterus of a mammal, and (c) carrying the transgenic mammal to full term. Finally, the invention relates to a method for the production of a transgenic plant.

Several documents are cited in the description. The disclosure content of these documents including manufacturers' instructions is incorporated by reference herein.

Due to the fact that the introduction of genetic modifications by means of genetic engineering are rare events both in prokaryots and eukaryots, selection methods suitable for the accumulation of clones in which the desired event has taken place are necessary. In principle, a distinction can be made between two possibilities: Positive selection can be used to select clones having a specific property. In contrast, with negative selection, cells lacking a specific property are accumulated. For positive selection, on the one hand, selection cassettes coding for a product allowing for survival in a deprivation medium are suitable. Alternatively, for example, expression cassettes can be used for positive selection the product of which inactivates a toxin contained in the medium and thus allows for survival of the genetically modified organism. In the case of negative selection, the product itself inhibits survival of the organism or the product expressed by the selection cassette converts, for example, a non-toxic precursor into a toxin which then kills the organism in question. With some enzymes both positive and negative selection is possible, depending on the substances present in the medium. For example, corresponding substances are known for enzymes of the nucleotide metabolism. There is, however, the problem that the relevant enzymes in eukaryotic cells are normally expressed in an endogenous manner. Thus, for the use of appropriate selection cassettes cells would have to be produced first, which lack the relevant enzymes (HPRT, APRT, TK, DHFR etc.). In fact, cells of that kind have been used in mutation research for years. Moreover, HPRT negative ES cells are already used for homologous recombination where the targeting constructs used for that purpose contain a HPRT expression cassette for positive selection. HPRT offers the advantage that the use of HAT (hypocanthine-aminopterin-thymidine) medium allows selection for the presence of a functional HPRT gene while the use of 6-thiguanosine or 8-azaguanosine allows selection against the presence of a functional HPRT gene.

Conventional selection systems are based essentially on the selectable properties which are mediated by proteins coded by nucleic acid sequences inserted into the cell. These foreign, i.e. exogenous proteins often have allergenic character and can induce a strong immune response in mammals, in particular in humans. Therefore, the use of these selection systems, for example in vectors for gene therapy or in the production of transgenic plants, is limited. Furthermore, the use of proteins encoded by resistance genes often runs the risk of resistance formation in prokaryots.

Due to the increasing importance of gene therapy in the treatment of diseases and due to the increasing use of transgenic plants in food production, there is an urgent need for selection methods that do not use the expression of foreign proteins in the genetically modified cells.

Thus, the problem of the present invention is the provision of a selection method which is safe for the environment and neither can mediate resistances in procaryots nor has an allergenic or immunostimulatory effect, in particular, on the human organism. This problem is solved by the provision of the embodiments characterised in the patent claims according to the invention.

Thus, the invention relates to a method for the production of a eukaryotic cell selectable by inactivation or reduction of an endogenous gene function, comprising the steps of (a) introduction of one or more vectors into the cell and (b) expression of a siRNA and preferably shRNA coded by the one or more vectors, directed against an endogenous selectable gene and inactivating same, said siRNA or shRNA being the transcription product of an RNAi selection cassette, the selection cassette comprising a section of at least 1-9 nucleotides of the transcribed region of the gene, said section being operatively linked to a promoter and a transcription termination signal.

The methods and individual method steps of the present invention can be carried out in vitro, in vivo and ex vivo. In a preferred embodiment, the cells used in the methods are non-human cells. In an alternatively preferred embodiment, the cells used are human cells, e.g. adult or embryonic stem cells, hematopoietic stem cells or lymphocytes.

The term “inactivation or reduction of an endogenous gene function” refers to the elimination or reduction of the gene function by at least 50%, preferably at least 75% and more preferably at least 80% such as e.g. at least 90% or 95% and up to 100%. Preferably, the values of the reduction are compared with a cell which is essentially identical to the cell containing the RNAi selection cassette, with the exception however that it does not contain the RNAi selection cassette. This cell can e.g. be a cell of the same cell line or a cell from the same tissue, preferably with the same differentiation status. According to the invention, the endogenous gene function is inactivated or reduced by expression of an RNAi comprising a short double stranded or complementary section of the endogenous gene. As a result, the protein biosynthesis of the protein encoded by the endogenous gene is considerably reduced or completely inhibited.

The term “selectable cell” refers to a cell that, in comparison with other cells, which are preferably present in the same cell population, exhibits properties which are advantageous or disadvantageous for the survival of this cell as compared to said other cells. On the basis of this advantage or disadvantage, the cell or the cells having the same selectable properties will be enriched or depleted in comparison to the other cells without the selectable properties. According to the invention, the selectable property can be a transiently or stably integrated property of the cell. According to the invention, it is preferably genetically integrated.

The term “introducing [ . . . ] in a eukaryotic cell” comprises all techniques for introduction of nucleic acid into eukaryotic cells which are known to the person skilled in the art. Said techniques include for example microinjection, transfection including electroporation, lipofection or calcium phosphate precipitation or any further introduction of a vector containing the nucleic acid to be inserted. A vector can be, for example, a viral vector such as e.g. an adenoviral vector or a lentiviral vector. It is, however, also possible that a single protein molecule mediates the introduction into the cell. Furthermore, transfection techniques are also known to the person skilled in the art which include the use of macromolecular polymers, e.g. fullerenes. Finally, the term “introduction” also refers to the ballistic methods known to the person skilled in the art which are used for the transfection of all eukaryotic cells, in particular however for the transformation of plant cells.

The RNAi selection cassette inserted into the cell can be inserted into the cell as RNA or DNA. Accordingly, the vector according to the invention refers to a DNA- or RNA-based vector. Preferred vectors are characterised in that they contain polymerase III dependent promoters such as, for example, H1 or U6 promoters or other polymerase III dependent promoters, e.g. 5S rRNA, snRNA and tRNA promoters (see Mittal, V. 2004: Improving the efficiency of RNA interference. Nature Reviews Genetics, 5: 355-365). Vectors containing inducible regulation sequences are further preferred. These include, for example, tetracycline-inducible or Ecdysone-inducible regulation sequences.

“Expression of a siRNA and preferably shRNA encoded by the [ . . .] more vectors” refers to the fact that the RNAi selection cassette, which according to the invention can be located on several vectors, leads to the transcription of a siRNA or shRNA. In the cases where the RNAi selection cassette is located on at least two vectors and where the parts of the cassette are flanked by recombinase detection sequences, a recombination of the separate nucleotide sequences is necessary before expression of the siRNA or shRNA.

Apart from a promoter sequence and a terminator sequence, the “RNAi selection cassette” comprises a nucleotide sequence with preferably 100% complementarity in the nucleic acid sequence to the endogenous selectable gene. In specific preferred embodiments lower complementarity is preferred. Thus, it can be guaranteed, for example, that a low degree of basal expression of the target gene further exists. In a preferred embodiment, the transcribed part of the RNAi selection cassette is transcribed in an arrangement of sense and antisense which is interrupted by a non-complementary region. This structure is called “small hairpin (sh) RNA” and contains sequences of the target mRNA as imperfect palindromic, wherein the two halves of the sense and antisense structure are separated by a short non-palindromic sequence. The non-palindrome sequence has a preferred length of 3 to 10, 10 to 100 or 100 to 1000 nucleotides. Short non-palindromic intermediary sequences with a length of 6 to 9 nucleotides are preferred, wherein the two nucleotides at the 5′ end of the non-palindromic sequence are preferably T's. It is also possible that the non-palindromic region contains one or more detection sequences of recombinases. For example, the loxP site with a length of 34 nucleotides or the frt site with a length of 48 nucleotides could be located in the non-palindrome region.

In another preferred embodiment, the interfering siRNAs are derived from longer precursor molecules which are degraded to siRNAs by the endogenously occurring enzymes (“dicer”) mentioned in this description below.

Thus, the method according to the invention makes use of a phenomenon known in the art as RNA interference (RNAi). RNAi is an endogenous cellular regulation mechanism causing, in eukaryots, the specific posttranscriptional inactivation of the expression of a target gene. Double-stranded so-called short interfering (si) RNAs, having an approximate length of 19 to 29 nt, are responsible for said activity (indication of length according to Mittal, V. 2004: Improving the efficiency of RNA interference. Nature Reviews Genetics, 5, 355-365), which are incorporated in an effector complex designated RISC(RNA induced silencing complex) which then mediates different functions such as degradation of the target mRNA or a translational suppression of the target mRNA. RISCs are ribonucleotide complexes containing different proteins, siRNA as well as the complementary mRNA, whereas it is as yet unclear how the degradation activity known as slicer is mediated. A single siRNA molecule can mediate the sequential degradation of several mRNA molecules in the RISC complex.

siRNA molecules are produced in vivo from longer double-stranded precursor RNAs by means of an RNAseIII known as dicer. Accordingly, transfected longer double-stranded RNAs specific for a certain target mRNA are degraded to biologically effective siRNAs. Another possibility of producing gene-specific siRNA molecules is the transfection of polymerase III-dependent expression vectors expressing so-called small hairpin (sh) RNAs. These shRNAs contain a 19 nt long part of the sequence of the target mRNA as imperfect palindrome, whereas the two halves of the repeat are separated by a short non-palindromic sequence. According to the invention there are, at the 3′ end of the construct to be expressed, preferably 5 consecutive thymidines which are recognised by the polymerase III as transcriptional termination signal. In accordance with the explanations above, in a preferred embodiment of the invention, a pol III promoter is used as promoter in step (a).

In vivo, corresponding transcripts form RNA double helices with an RNA loop at one end and a 3′ overlap with a length of two Ts at the other end. Due to the effect of dicer, the loop is removed so that a functional siRNA molecule is formed which is able to mediate the RISC-mediated inactivation of the target mRNA.

Experiments carried out in mammalian cells suggest that there are at least two responses to dsRNA, an unspecific and a specific dsRNA response, which are competing for the dsRNA. Unspecific RNAi effects are said to be due to, amongst others, the presence of an antiviral mechanism which is common in mammalian cells and is known as interferon response. Longer dsRNA molecules induce the unspecific dsRNA response, if they have a length of at least 30 base pairs. In this context, cellular proteins recognise the dsRNA and initiate a general inhibition of the cellular translation (Terenzi et al, 1999; Williams, 1999). This leads to an unspecific reduction of gene expression. In this context, the dsRNA activates, amongst others, two enzymes: PKR, which, in its active form, phosphorylates the translation initiation factor elF2a, thus leading to the termination of protein synthesis, and 2′, 5′ oligoadenylate synthetase which forms a molecule that activates RNase L, a non-specific enzyme degrading mRNAs (Elbashir et al., 2001).

Due to the likelihood of a cellular interferon response increasing with increasing length of the small hairpin (sh) RNA, it is preferred that the small hairpin (sh) RNA transcribed by the RNAi selection cassette has a length of up to 200 nucleotides. Even more preferred, however, are lengths of up to 50 nucleotides and up to 30 nucleotides and most preferred are small hairpin (sh) RNA lengths of 19 nucleotides, 20 nucleotides, 21 nucleotides, 22 nucleotides, 23 nucleotides, 24 nucleotides, 25 nucleotides or up to 29 nucleotides. Preferably, the section of the RNA complementary to the endogenous gene has a length of 19 nucleotides, 20 nucleotides, 21 nucleotides, 22 nucleotides, 23 nucleotides, 24 nucleotides or 25 nucleotides.

The term “section of at least 19 nucleotides” refers to the fact that the RNAi selection cassette transcribes a siRNA or an shRNA containing a section with a length of at least 19 nucleotides, wherein said section is complementary to a transcribed section of the endogenous cellular gene. The siRNA or shRNA can have a length of up to 200 nucleotides in total. In this context, however, the number of nucleotides identical or essentially identical to the target sequence via a consecutive section (complementary region) is preferably 19 to 50 nucleotides, more preferably 19, 20, 21, 22, 23, 24 or 25 nucleotides. Preferably, the section of the shiRNA/shRNA which is complementary to the endogenous gene thus has a length of 19 to 50 nucleotides and the lengths of 19 nucleotides, 20 nucleotides, 21 nucleotides, 22 nucleotides, 23 nucleotides, 24 nucleotides or 25 nucleotides are most preferred, wherein particularly in the case of the shRNA, further nucleotides can be present which form the non-palindromic region. According to the definition, the non-palindromic region is located between the anti-parallel section which is formed from a nucleotide section in sense orientation and a nucleotide section in antisense orientation. The term “essentially identical” relates to sections with incomplete complementarity. In specific preferred embodiments, a low complementarity is preferred. A lower complementarity can, for example, ensure that a low degree of basal expression further exists. Preferably, the essentially identical sections within the RNAi or shRNA have a sequence identity to the target sequence of at least 90%, at least 91%, at least 92%, at least 93%, at least 94%, at least 95%, at least 96%, at least 97%, at least 98% or at least 99%. Essentially identical sections with a 100% sequence identity to the target sequence are most preferred.

The term “operatively linked to a promoter and a transcription termination signal” refers to the fact that a transcription of the sense and antisense structure described in detail above into RNA is possible which is mediated by the promoter and is terminated by the transcription termination signal. For this purpose, preferred promoters are, as mentioned above, pol III promoters (Review: Paule M R, White R J (2000) Survey and summary: transcription by RNA polymerases I and III. Nucleic Acids Res. 28(6); 1283-1298) e.g. H1 Promoter (Brummelkamp T R, Bernards R, Agami R. (2002): A system for stable expression of short interfering RNAs in mammalian cells. Science 296: 550-553), U6 Promoter (Sui G, Soohoo C, Affar el B, Gay F, Shi Y, Forrester W C, Shi Y (2002): A DNA vector-based RNAi technology to suppress gene expression in mammalian cells. Proc. Natl. Acad. Sci. USA 99(8): 5515-5520; Paul C P, Good P D, Winer I, Engelke D R (2002): Effective expression of small interfering RNA in human cells, Nat. Biotechnol. 20(5): 505-508), 5S rRNA and tRNA promoters, but also less preferably polymerase II promoters such as e.g. the CMV promoter (Xia, H, Mao Q, Paulson H L, Davidson B L (2002): siRNA-mediated gene silencing in vitro and in vivo. Nat. Biotechnol. 20(10): 1006-1010; Shinagawa T, Ishii S (2003): Generation of Ski-knockdown mice by expressing a long double-strand RNA from an RNA polymerase II promoter. Genes Dev. 17(11):1340-1345). Preferred termination sequences for polymerase III-dependent promoters are at least 4, preferably 5 consecutive thymidine (T) downstream of the section of the RNAi construct to be transcribed (Review: Paule M R, White R J (2000) Survey and summary: transcription by RNA polymerases I and III. Nucleic Acids Res. 28(6):1283-1298). An extension by further consecutive Ts is also possible and is comprised by the present invention.

The presented results show that the method according to the invention using, in a preferred embodiment, HPRT-RNAi constructs (see below) is suitable for lowering the expression of the target gene in cells to such an extent that the cells can be selected without undue effort. In the case of the preferred embodiment mentioned, the cells develop a resistance against 6-thioguanosine and 8-azaguanosine and, at the same time, become sensitive to HAT medium. Thus, the observed properties of the transfected cells present a phenocopy of cells produced in a free-of-protein manner, which exhibit a reduced/inactivated expression of the target gene.

In the following, advantages of the invention are described in more detail by means of the preferred embodiment HPRT-RNAi selection cassette. According to the invention, however, the method of the invention can also be extended to any eukaryotic selectable gene functions without undue effort being required for this purpose. According to the teaching of the invention and on the basis of his general knowledge the person skilled in the art can select an appropriate gene/protein, determine a nucleotide sequence against which the siRNA/shRNA is directed and carry out the method as selection method. Further preferred target genes/target proteins are described in detail below.

A further advantage of the system according to the invention is that the phenotype, for example RNAi-mediated HPRT-deficiency of cells, can be reverted by removal of the RNAi selection cassette, e.g. by means of recombinase-mediated deletion or by homologous recombination. In the case of our example, this leads to the cells again becoming HAT-resistant and sensitive for 6-thioguanosine and 8-azaguanosine. Accordingly, the cells stably transfected with HPRT-RNAi constructs could be reconverted into HAT-sensitive cells, if the relevant construct is removed from the genome of the cells, e.g. by use of appropriate recombinase systems.

In comparison to conventional selection systems, RNAi-based selection systems also differ in an advantageous manner in that they do not require expression of proteins. Thus, they are to be classified as non-allergenic, which should be an advantage for the production of transgenic plants or for use in vectors for gene therapy. Furthermore, such cassettes have the advantage that they cannot mediate resistance or sensitivity in prokaryots, due to them lacking the RNAi mechanism. Moreover, the shRNAs required can be produced species-specific so that a potential horizontal gene transfer e.g. from an apathogenic species to a pathogenic species should have no consequences.

Since one copy of the HPRT-RNAi construct (which is preferred according to the invention) suffices for the effects observed, appropriate cassettes can be used for the introduction of any modifications by genetic engineering into cells, for which, so far, other selection cassettes have been used (e.g. neo, hygro or puro selection cassettes), and thus can also be used for the production of genetically modified animals or plants. At the same time, RNAi-based selection cassettes are significantly smaller (<200 bp) than commonly used selection cassettes that are based on the expression of proteins. This shortening is particularly due to the use of RNAIII-dependent promoters. The H1 promoter, for example, has a length of only 100 bp, whereas common selection cassettes with promoters based on RNAII polymerase are often significantly longer due to the promoter sequences and polyA signals (>1000 bp) and often cause problems during cloning due to their lengths. Furthermore, even target mRNAs are considered for which only substances for the selection against the product exist (e.g. dihydrofolate reductase, Table 1).

Since one copy of the HPRT-RNAi construct suffices for the mediation of resistance against 6-thioguanosine and 8-azaguanosine, it is furthermore possible to use RNAi-based selection cassettes in conventional and conditional gene trap vectors.

A disadvantage of the RNAi technique is the fact that it does not work in prokaryots. Nevertheless, the use of appropriate cassettes in the production of targeting constructs by means of homologous recombination in bacteria is possible, if it is combined with a second selection marker (e.g. β-lactamase) in a cassette. The prokaryotic selection marker could then be removed by means of recombinase-mediated deletion so that in a construct produced in that manner, the RNAi cassette effective in eukaryots would be maintained and could be used e.g. for the production of transgenic animals.

In a preferred embodiment of the invention, the method according to the invention comprises the further step (c) of the expression of a recombinase, wherein the RNAi selection cassette is formed after homologous recombination, wherein before the recombination between the recombination precursors, between promoter and RNAi selection cassette or within the RNAi selection cassette a 5′ and a 3′ recombinase recognition sequence is located and between those a separating nucleotide sequence is located, wherein the separating nucleotide sequence contains a transcription termination signal and wherein a homologous recombination at the recombinase recognition sequences takes place as a consequence of the expression of the recombinase. The separating nucleotide sequence fulfills the function to initially inhibit the transcription of an RNAi or shRNA. Accordingly, it is possible that instead of the known transcription termination signal, for example, cellular nucleotide sequences with similar function or effect are contained.

In this preferred embodiment of the invention, a non-functional RNAi selection cassette is introduced into the cell. This inactive state of the RNAi selection cassette is ensured by the presence of separating nucleotide sequences within the RNAi selection cassette, wherein the separating nucleotide sequences comprise a transcription termination signal. Since the separating nucleotide sequences are flanked by recombinase recognition sequences, the separating nucleotide sequences can be removed by expression of the corresponding a recombinase for the recognition sequences and, thus, a functional RNAi selection cassette can be formed. As described further below, the recombinase recognition sequences can be located, for example, between the first and the second sequence of the inverted sequence repeat of the shRNA. It is furthermore possible to distribute the RNAi selection cassette on several vectors and to combine it only after a recombination event, i.e. to put it into a functional state.

Preferably, recombinase recognition sequences are selected from the group consisting of loxP, frt, attb-attp as well as mutated forms thereof. By expression of the relevant recombinase Cre, flp, phiC31, the region between the recombinase recognition sequences is deleted, so that the RNAi construct is reconstituted and activated and can thus be selected for the relevant deletion.

In a more preferred embodiment of the method according to the invention, the recombinase recognition sequences are located between the first and the second sequence of the inverted sequence repeat of the shRNA. In this context, the term “inverted sequence repeat” relates to the above-described structure of nucleotide sequences in “sense” and “antisense” orientation. By expression of the relevant recombinase, the region between the recombinase recognition sequences is deleted so that the RNAi construct is reconstituted, wherein the remaining recombinase recognition sequence forms the loop, i.e. the non-palindromic section in the expressed shRNA molecule. With the RNAi construct activated in said manner it is thus possible to select for the relevant deletion. In case the “inverted sequence repeats” are located in two separate positions of a chromosome, it is possible to delete the chromosome region flanked by the halves of the RNAi construct by means of expression of the appropriate recombinase, wherein the reconstitution of the RNAi construct allows for a selection for the deletion which has taken place.

In another more preferred embodiment of the method according to the invention, (i) the promoter, the 5′ recombinase recognition sequence and the first sequence of the inverted sequence repeat of the shRNA and (ii) the 3′ recombinase recognition sequence and the second sequence of the inverted sequence repeat of the shRNA are located on different vectors. In this method, both halves, i.e. the promoter with a recombinase recognition sequence and the second recombinase recognition sequence with the transcribed RNAi sequence effective during RNA interference, are located in trans, i.e. on different molecules, preferably different vectors or chromosomes. The effect of the relevant recombinase after expression of same leads to intermolecular recombination, preferably interchromosomal recombination, i.e. translocation of the relevant sections of the chromosomes, and to reconstitution of a functional RNAi construct, so that selection for the intermolecular recombination is possible.

In another preferred embodiment the method according to the invention comprises the further step (d) of culturing and enrichment of cells containing or expressing the RNAi selection cassette or (d′) of culturing and enrichment of cells not containing and not expressing the RNAi selection cassette. In case the method according to the invention does not comprise, for example, the step (c), the steps (d) and (d′) mentioned herein are to be understood as step (c) and (c′) accordingly. The same applies to the further method steps mentioned in the application and the combinations thereof. The variant (d′) of culturing and enrichment of cells not containing and not expressing the RNAi of selection cassette as second method step can be used in cases when selection is to take place against the expression of the RNAi construct. Advantageously, this system can be used when selection against the random integration of a genomic construct is to take place. An RNAi selection cassette is constructed in a manner that the desired DNA to be inserted into the genome is flanked by sequences homologous to the target sequence. At the one end of at least one flanking sequence the RNAi selection cassette is cloned which interferes with the expression of a gene essential for the cell. This essential gene can code, for example, for a structure protein such as actin or an enzyme such as the polymerase II. Upon integration into the desired region the RNAi selection cassette is lost so that there is no interference with the expression of the essential gene. If, on the other hand, random integration takes place, there is a great chance of the RNAi construct also being integrated into the genome. In this case, an inhibition or reduction of the essential gene function takes place resulting in the dying of cells or in disadvantages as to the growth of cells.

In a preferred embodiment of the invention, the selectable gene is positively and/or negatively selectable. According to the invention, “positively selectable” relates to the enrichment of cells exhibiting RNAi expression. According to the invention, “negatively selectable” relates to the enrichment of cells not exhibiting an RNAi expression.

In another preferred embodiment of the invention, the endogenous selectable gene encodes a product capable of converting a non-selecting precursor of a substance A into a product B with selecting or selectable properties and wherein the cell is cultured in the presence of the substance A.

In another preferred embodiment of the invention, the non-selecting substance A is a non-toxic precursor of a toxin and the product B is a toxin. The term toxin refers to a chemical compound having an inhibiting effect on the division or the growth of cells. Examples of toxins are nucleotide analogues leading to chain termination during the synthesis of DNA sequences. Further examples are mentioned in Table 1. According to the technical teaching of the invention, the expression of the RNAi or shRNA described above in detail leads to cells becoming sensitive to specific substances, for example to the substances listed in Table 1, wherein sensitivity refers to the fact that cells stop growing or die.

Constructs to be used in a similar manner to the HPRT-RNAi construct described herein are also available for other endogenous mRNAs, for other RNAs, the products of which can be selected for or against by means of substances added in an exogenous manner (e.g. thymidine kinase and APRT). An overview of the genes/gene products particularly preferred and the substances selecting same is given in Table 1.

TABLE 1
Effect of different selection media on wild type cells (wild type),
on cells expressing RNAi-constructs (RNAi) directed against
HPRT, APRT thymidine kinase or DHFR as well as on cells
in which the corresponding genes are inactivated on the
chromosomal level (knock out). Survival of the cells in the
respective selective conditions is marked +; cell death in the
respective selective condition is marked −.

| Gene | | | | |
|---|---|---|---|---|
| (HomoloGene | | | | |
| entry) | | | | |
| HomoloGene, | | | | |
| build 36, last | | | | |
| update | | | | |
| May 25, 2004 | selection medium | wild type | RNAi | knock out |
| HPRT | HAT | + | − | − |
| (162) | 8-azaguanosine | − | + | + |
| | or | | | |
| | 6-thioguanosine | | | |
| APRT | ALASA | + | − | − |
| (413) | (alanosin/azaserine/ | | | |
| | adenine) | | | |
| | diaminopurine, | − | + | + |
| | 8-azaadenine or | | | |
| | 2-fluoroadenine | | | |
| thymidine | HAT or CHAT | + | − | − |
| kinase | | | | |
| (2446) | | | | |
| | trifuorothymidine or | − | + | + |
| | bromodesoxyuridine | | | |
| DHFR | methotrexate | + | − | − |
| (619) | | | | |

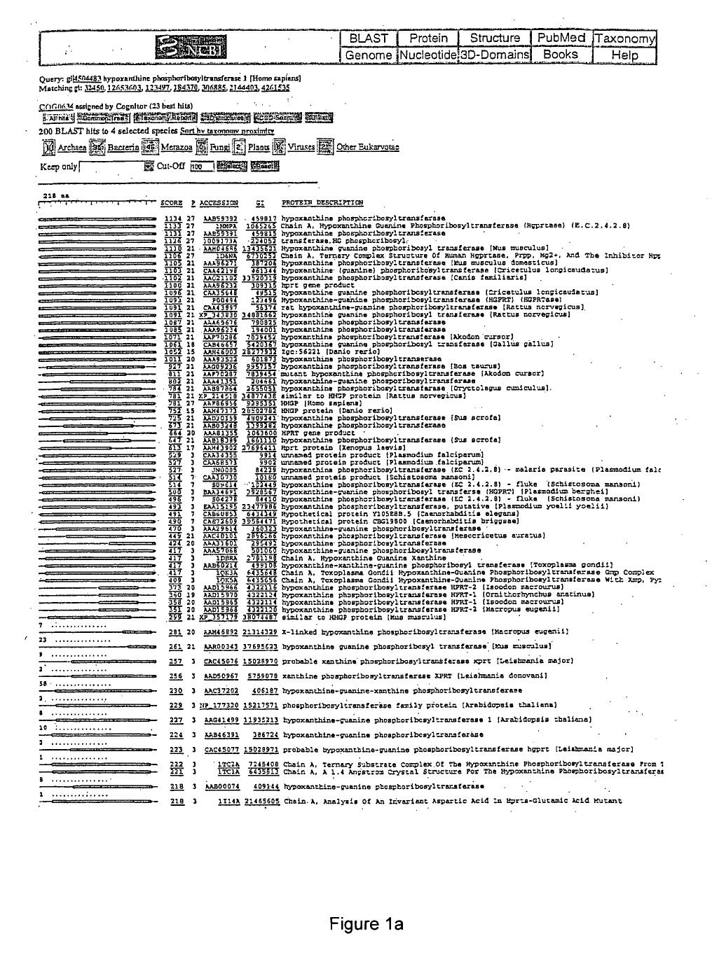

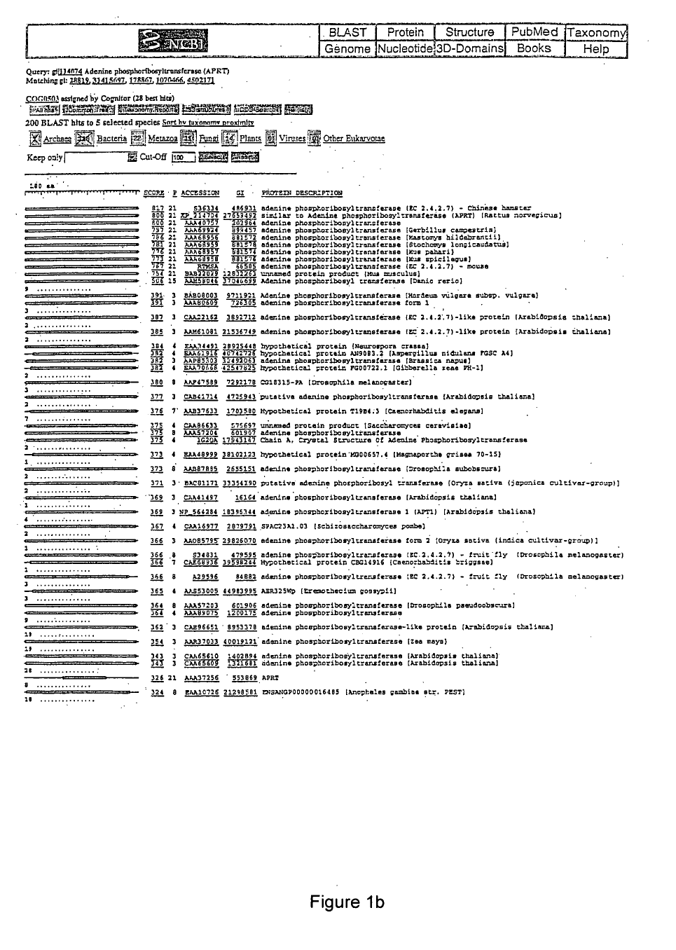

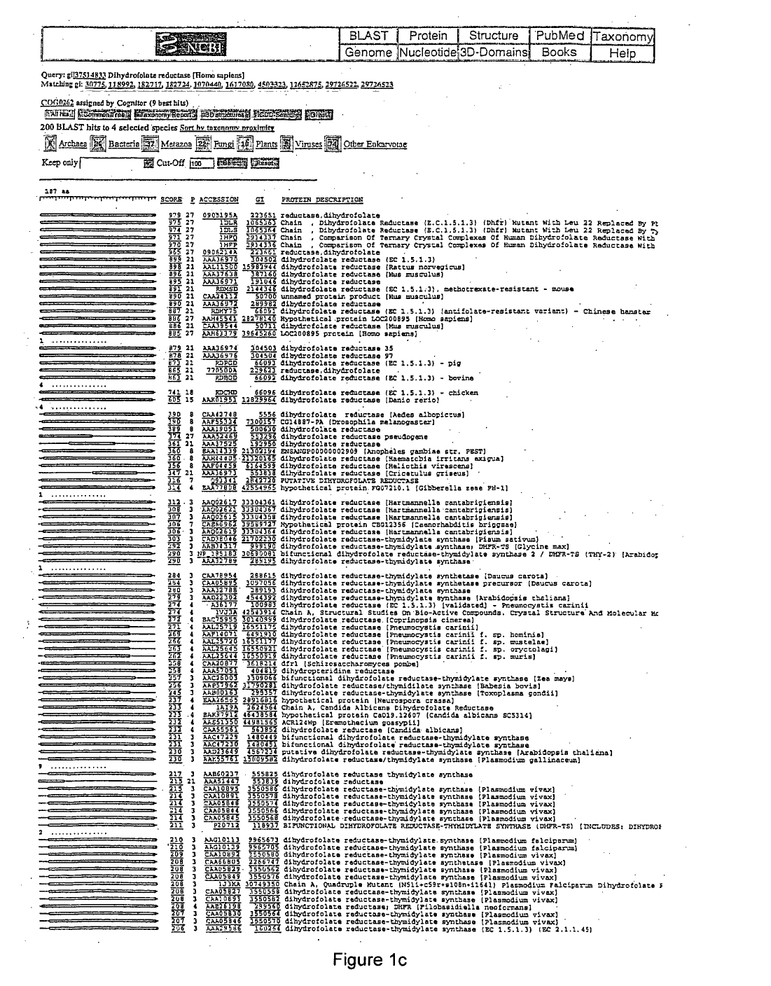

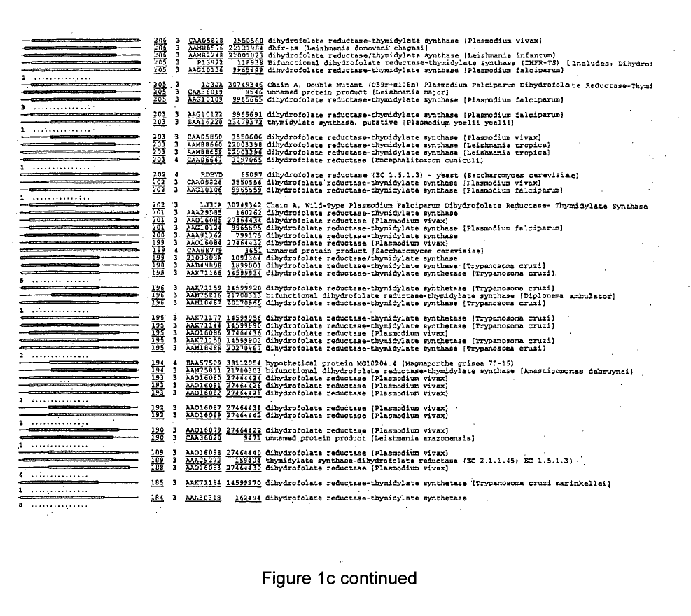

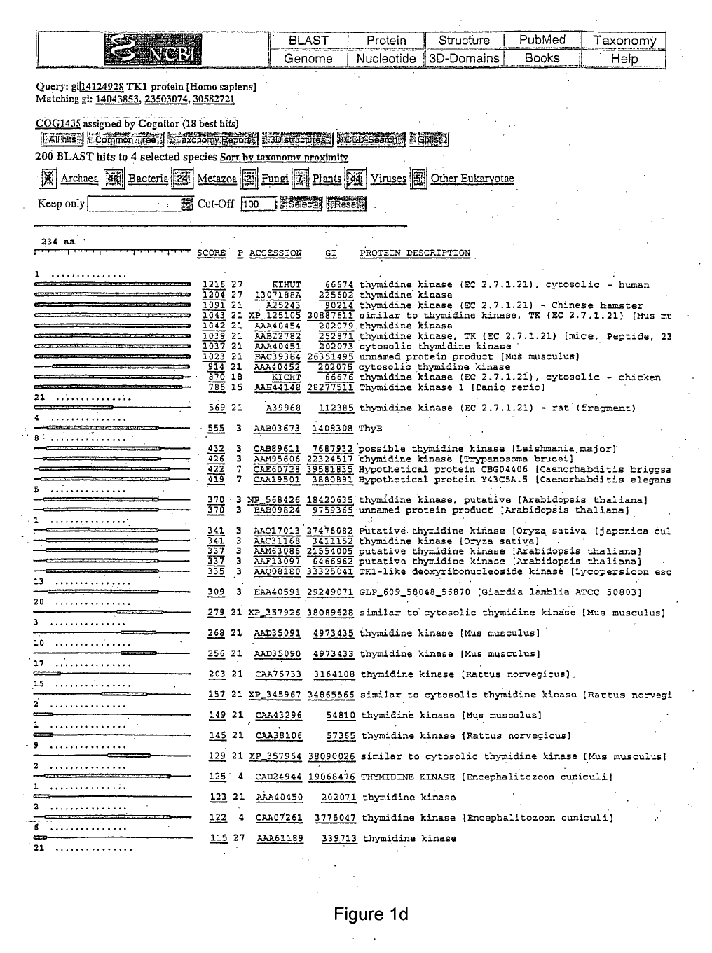

According to the above considerations, in a particularly preferred embodiment of the invention, the selectable gene is APRT, DHFR or TK. In a further particularly preferred embodiment of the invention, the selectable gene is HPRT, as mentioned above. The above-mentioned genes comprise all known allelic variants of the genes, in all species. The genes listed in the HomoloGene database of the NCBI (HomoloGene build 36, last update May 25, 2004) are particularly preferred: HPRT HomoloGene: 162, APRT HomoloGene: 413, thymidine kinase HomoloGene: 2446, DHFR HomoloGene: 619) which are listed in FIG. 1a-d. The accession numbers of the sequences deposited in the gene library are indicated in the Figures. In this context, HPRT represents hypoxanthine guanine phosphoribosyltransferase (HPRT; EC 2.4.2.8) which plays an important role in the metabolism of the purine bases hypoxanthine and guanine and plays a particularly prominent role in the fusion of myeloma cells and B-cells for the production of hybridomas (see Harlow and Lane, “Antibodies, A Laboratory Manual”, CSH Press, Cold Spring Harbor, 1998).

According to the invention, an embodiment in which 6-thyoguanosine or 8-azaguanosine are used for selection is also particularly preferred. In a further particularly preferred embodiment of the invention ALASA (alanosine/azaserine/adenine), diaminopurine, 8-azaadenine or 2-fluoroadenine, HAT or CHAT, trifluorothymidine or bromodesoxyuridine or methotrexate are used for selection.

In another preferred embodiment of the invention, at least one additional nucleotide sequence is introduced into the cell together with or subsequently to the RNAi-selection cassette or its recombination precursor. Preferably, the homologous genomic nucleotide sequences are such that a homologous recombination into the cellular genomic DNA is possible. Preferably, the flanking 5′ and 3′ located homologous genomic nucleotide sequence is up to 100 bp long, more preferred up to 1.000 bp long, still more preferred up to 10.000 bp long and most preferred up to 200.000 bp long. Ideally, the flanking nucleotide sequence has 100% sequence identity, however, the use of flanking sequences with less sequence identity are also conceivable. Accordingly, “homologous nucleotide sequences” refer to nucleic acid segments which show a sequence identity over the entire sequence length of at least 50%, preferably at least 70%, more preferably at least 90%, still more preferably 95% and particularly preferred at least 100% sequence identity. Preferably, sequence identity is determined by FASTA, BLAST (Basic Local Alignment Search Tool) or Bestfit algorithms of the GCG sequence analysis programme (Wisconsin Sequence Analysis Package, Version 8 for Unix, Genetics Computer Group, University Research Park, 575 Madison, Wis. 53711). If Bestfit is used, parameters are preferably adjusted in a way that the identity percentage is calculated over the entire length of the reference sequence and homology gaps of up to 5% of the total of nucleotides are allowed. If Bestfit is used, the so-called optional parameters preferably are set at the pre-selected values.

In a further preferred embodiment of the invention, the at least one additional nucleotide sequence are two nucleotide sequences with a homology to cellular genomic nucleotide sequences which are flanking the RNAi-selection cassette or its recombination precursor at the 3′ and 5′ ends. The additional nucleotide sequence can be a gene or a gene fragment. Preferably, the additional nucleotide sequence is an endogenous or exogenous nucleotide sequence. The term “exogenous nucleotide sequence” refers to a nucleotide sequence which was introduced from outside and has no equivalent within the cell. Examples of exogenous sequences are bacterial genes or fusion constructs. Accordingly, the term “endogenous nucleotide sequence” refers to a nucleotide sequence having a equivalent within the cell.

In a more preferred embodiment of the invention, the additional nucleotide sequence introduced into the cell is located on the vector which contains the RNAi selection cassette and the additional nucleotide sequence is enclosed by the 5′ and 3′ flanking sequences having homology to the cellular nucleotide sequences. Flanking sequences which are necessary for homologous recombination are preferably up to 100 bp long, more preferably up to 1.000 bp long, still more preferably up to 10.000 bp long and most preferably up to 200.000 bp long.

In a further particularly preferred embodiment of the invention, the additional nucleotide sequence introduced into the cell (in all embodiments of the invention, e.g. a gene or an expressible coding sequence which encodes a protein or peptide having a desired property) is located on a separate vector and, 5′ and 3′ of the additional nucleotide sequence, nucleotide sequences showing homology to cellular genomic nucleotides sequences are present.

According to a further embodiment of the method of the invention, it is further preferred that the additional nucleotide sequence introduced into the cell is introduced subsequently into the cell and that the additional nucleotide sequence is flanked 5′ and 3′ by homologous genomic nucleotide sequences which allow a recombination of the additional nucleotide sequence into the chromosomal position of the RNAi expression cassette. Preferably, the recombination into the position of the RNAi expression cassette inhibits further RNAi expression, i.e. the RNAi expression cassette is removed by homologous recombination. More preferably, the flanking nucleotide sequences are designed in such a way that the RNAi expression cassette is completely removed from the chromosome. To this aim, the flanking nucleotide sequences of the RNAi expression cassette and of the nucleotide sequence subsequently introduced are identical or they overlap.

In a further particularly preferred embodiment of the invention, the RNAi selection cassette and/or the additional nucleotide sequence introduced into the cell is, in addition, flanked by recombinase recognition sequences. The recognition sequences loxP, frt, attb-attp and mutated forms of loxP, frt, and attb-attp recognition sequences or the like for the recombinases cre, flp and phiC331 or other recombinases (there will be further sequences) are preferred. Provided the constructs have structures which are homologous to a target gene, the homologous structures are preferably located in terminal position with respect to the recombinase recognition sequences so that the latter are maintained in the construct integrated into the genome when a recombination event occurs.

According to the invention, a method wherein the additional nucleotide sequence introduced into the cell is a gene or a fragment thereof is also particularly preferred.

According to another embodiment of the method of the invention, it is particularly preferred that the gene is a mutant or allelic variant of an endogenous gene of the cell. The term “allelic variant” means that the allelic variant differs in at least one nucleotide from the corresponding gene. Moreover, the method of the invention comprises also embodiments in which an orthologous gene is introduced into the cell. If the cell is a human cell, particularly preferred embodiments are embodiments in which the gene is derived from mouse. If the cell is a murine cell, particularly preferred embodiments are embodiments in which the gene is derived from human. The gene can be inserted on the same chromosome and at the position of the corresponding cellular gene or at a different position, for example, within the same chromosome or on another chromosome.

In a further particularly preferred embodiment of the invention, the gene is an exogenous gene. The term “exogenous gene” comprises genes which have no equivalent within the cell. These include, for example, bacterial genes or fusion constructs, which are to be introduced into the cell.

In another preferred embodiment of the invention, the method of the invention further comprises the following steps: (e) inactivation or reduction of the RNAi expression; and (f) selection and enrichment of cells which do not exhibit RNAi-based inactivation or reduction of the endogenous selectable gene. Inactivation can take place, for example, by recombination into the position of the RNAi selection cassette. The RNAi selection cassette might also be controlled by a conditionally effective promoter or by appropriate regulatory elements known to the person skilled in the art. Regulation via tetracycline or ecdyson regulatable control elements, for example, is possible.

In principle, this preferred embodiment of the method according to the invention can be applied to all genes/gene products which can be selected subsequent to inactivation or significant reduction of the RNAi. An excellent example of this kind of application is the use of the HPRT gene as target of the interference. HPRT-based selection can be used, in particular, for the production of allelic series with diverse mutations of the same allele. To this aim, the corresponding allele is first provided with the HPRT-RNAi construct by means of homologous recombination and selected with 6-thioguanosine or 8-azaguanosine. Subsequently, genomic constructs containing the desired mutations but no selection marker are transfected into the corresponding 6-thioguanosine or 8-azaguanosine selected clones. Homologous recombination clones having the desired mutations can then be selected by means of HAT selection. In principle, the 6-thioguanosine or 8-azaguanosine selected clones can be expanded and cells thereof can be transfected with the desired genomic constructs and, at the same time, cells can be selected using HAT. In this way it is possible to produce allelic variants of a gene which differ from each other only in one single nucleotide or one single codon. It is understood that also variants can be produced which differ in more than one nucleotide or codon. Embodiments in which allelic variants are produced by means of serial and alternating transfection of a HPRT-RNAi selection cassette and of genomic constructs are less preferred but also comprised within the scope of protection of the invention.

In a particularly preferred embodiment of the invention, the inactivation of the RNAi expression is a recombinase-mediated deletion and comprises the step of expression of a recombinase. To this aim, the RNAi selection cassette is, for example, flanked 5′ and 3′ by recognition sequences of a recombinase.

In connection with the discussed preferred embodiment of the method according to the invention described herein, the RNAi selection cassettes described and, particularly, a HPRT-RNAi selection cassette are advantageous for the so-called recombinase-mediated cassette exchange (RMCE) (see EP-A1-0939120 and Bode, J., Schlake, T., Iber, M., Schubeler, D., Seibler, J., Sneshkov, E., Nikolaev, L. (2000):

The transgeneticists' toolbox: novel methods for the targeted modification of eukaryotic genomes. Biol. Chem. 381 (9-10):801-813)) whereby an RNAi selection cassette flanked by two different recombinase recognition sequences (e.g. a wild type and a mutated recognition sequence of a recombinase (cre, flp, phiC31 or others) is, first, homologously recombined into a locus and, using the corresponding recombinase, can then be exchanged for any sequence that is flanked by the same recognition sequences. This has the advantage that the same cassette can be used to select first for the integration of the same and, subsequently, for the RMCE event. In addition, the small seize of the cassette may be advantageous, since the efficiency of recombinases depends on the distance of their recognition sequences.

In a further particularly preferred embodiment and as mentioned above, the endogenous selectable gene is HPRT and the selection of cells which do not exhibit RNAi-based inactivation of the endogenous selectable gene takes place in the presence of HAT medium (see Table 1).

According to the invention, a further preferred embodiment relates to a method wherein the vector contains an additional cassette which, upon random integration into a cellular gene, leads to the inactivation thereof and, thus, serves as gene trap (Stanford, W. L., Cohn J. B., Cordes, S. P. (2001): Gene-trap mutagenesis: past, present and beyond. Nat. Rev. Genet. 2(10):756-768).

The application of the method described is not limited to cells of mammals, but can be applied to cells from any organisms in which RNAi is possible. Thus, the system can be applied to most eukaryotes. APRT-RNAi constructs, for example, might be used in plants. Here, the RNAi approach offers the same advantage as in mammalian cells in that wild type cells can be used, whereas the APRT selection systems available to present require plant cells without a functional APT1 gene, which are available only from few species. Since, as in the HPRT-RNAi system described above, positive- and negative-acting selection agents are known, this might offer the advantage that in seed production subject to open-air applicability of the system, it might be possible to select also for the wild type and, thus, it would be possible to produce seed of varietal purity without contamination of transgenes.

According to the above explanations, in another preferred embodiment of the invention, the eukaryotic cell is a plant cell. Transfer of the vector is carried out according to methods which are known to the person skilled in the art, preferably using plasmids, in particular plasmids which ensure stable integration of the DNA molecule into the genome of transformed plant cells, for example binary plasmids or Ti-plasmids of the Agrobacterium tumefaciens system. Apart from the Agrobacterium system, other systems for the introduction of DNA molecules into plant cells can be used, such as the so-called biolistic method or the transformation protoplasts (cf. Willmitzer, L. (1993), Transgenic Plants, Biotechnology 2; 627-659 for a survey). Methods for the transformation of monocotyledonous or dicotyledonous plants are described in the literature and are known to the person skilled in the art.

In order to ensure the expression of RNAi expression cassettes in plant cells, these can, in principle, be placed under the control of any promoter that is functional in plant cells. Generally, the expression of said RNAi selection cassette and of the additional nucleotide sequence can take place in any tissue of a plant regenerated from a transformed plant cell and at any time, preferably, however, it will take place in tissues in which a modified capability of forming and using specific proteins is advantageous either for the growth of the plant or for the formation of substances within the plant. In this case said specific protein is encoded by the additional nucleotide sequence introduced into the plant cell.

The method of the invention can be used to produce transgenic plants which express specific proteins. This can be the over-expression of an endogenous protein or the expression of a foreign protein. These so-called foreign proteins may be encoded, for example, by specific alleles of a gene. The technical teaching of the invention comprises both the additional expression of an allele as well as “knock-out” methods.

In principle, the transgenic plant cells may be cells of any plant species. Cells of monocotyledonous as well as of dicotyledonous plant species are of interest, in particular cells of starch storing plants or agricultural crop plants such as rye, oat, barley, wheat, potato, maize, rice, pea, sugar beet, tobacco, cotton, vine, tomato etc., or cells of ornamental plants.

In a more preferred embodiment of the invention, the plant cell is a protoplast.

In a further preferred embodiment of the invention, the eukaryotic cell is a vertebrate cell or an invertebrate cell. Particularly preferred invertebrates are selected from the group consisting of C. elegans and D. melanogaster. Particularly preferred vertebrates are selected from the group consisting of Danio rerio, human, mouse, rat, pig, bovine and primate.

In a particularly preferred embodiment of the invention, the vertebrate cell is a mammalian cell. In another particularly preferred embodiment the mammalian cell of the invention is the cell of a non-human mammal.

In a particularly preferred embodiment of the invention, the mammalian cell is a cell obtainable from mouse, rat, pig, bovine or primate. Preferably, the primate is human.

In a further particularly preferred embodiment of the invention, the cell is an embryonic stem cell, an adult stem cell, a hematopoietic stem cell, a somatic cell or a cell of an established cell line.

According to the technical teaching of the present invention, in a preferred embodiment, the cell of the invention is a cell outside the human or animal organism. Such a cell can be present, for example, in a cell cluster with other cells in culture. Said cell cluster can, for example, be a tissue. In another preferred embodiment of the invention, the cell of the invention is part of a living organism. Preferably, said living organism is a non-human mammal.

Further, the invention relates to a eukaryotic cell comprising an RNAi selection cassette directed against an endogenous selectable gene and inactivating or reducing its function, wherein the RNAi selection cassette comprises a section of the gene with a length of at least 19 nucleotides which is operatively linked to a promoter and a transcription termination signal.

In a preferred embodiment of the invention, the endogenous selectable gene is selected from the group consisting of HPRT, APRT, DHFR or TK. The afore-mentioned genes comprise all known allelic variants in all species. The genes listed in the HomoloGene NCBI database (HomoloGene build 36, last update May 25, 2004) are particularly preferred: HPRT HomoloGene: 162, APRT HomoloGene: 413, thymidine kinase HomoloGene: 2446, DHFR HomoloGene: 619) (see FIG. 1a-d).

In a further preferred embodiment of the invention, the RNAi selection cassette is stably integrated into the genome of the cell.

Another preferred embodiment of the invention relates to a cell which is a plant cell. In principle, transgenic plant cells of the invention can be cells of any plant species. Cells of monocotyledonous as well as of dicotyledonous plant species, in particular cells of starch storing plants or agricultural crop plants such as e.g. rye, oat, barley, wheat, potato, maize, rice, pea, sugar beet, tobacco, cotton, vine, tomato, etc., or cells of ornamental plants are of interest.

In a particularly preferred embodiment of the invention, the plant cell is a protoplast.

Furthermore, the invention relates to a transgenic plant containing the plant cell of the invention. Such plants can be produced, for example, by regeneration from plant cells of the invention using methods known to the person skilled in the art. Subject matter of the invention is further propagating material from plants of the invention which contains cells according to the invention. Said propagating material includes, for example, cuttings, seeds, fruits, roots, tubers, seedlings, etc.

In another preferred embodiment of the invention, the cell is a vertebrate cell or invertebrate cell. Particularly preferred invertebrates are selected from the group consisting of C. elegans, D. melanogaster. Particularly preferred vertebrates are selected from the group consisting of Danio rerio, mouse, human.

In a more preferred embodiment of the invention, the vertebrate cell is a mammalian cell.

According to the invention, in a particularly preferred embodiment, the mammalian cell is a cell obtainable from human, mouse, rat, pig, bovine or primate is. In a particularly preferred embodiment, the primate is human.

In another particularly preferred embodiment, the mammalian cell of the invention is the cell of a non-human mammal.

In a further particularly preferred embodiment of the invention, the cell is an embryonic stem cell, an adult stem cell, a hematopoietic stem cell, a somatic cell or a cell of an established cell line.

According to the technical teaching of the invention, in a preferred embodiment, the cell of the invention is a cell outside the human or animal organism. Such a cell can be present, for example, in a cell cluster with other cells in culture. Said cell cluster can for example be a tissue. In another preferred embodiment of the invention, the cell of the invention is part of a living organism. Preferably, said living organism is a non-human mammal.

The invention further relates to a method for the production of a transgenic animal, wherein (a) cells are treated according to one or more steps of the method of the invention and (b) a viable organism is grown from the cells. Said method is particularly suitable for the production of transgenic vertebrates and invertebrates.

The RNAi selection cassettes used in the method of the invention could also be used in a particularly advantageous way for the production of transgenic mice, preferably by means of ES cell transfection, as well as for genetic modifications in embryonic stem cells by means of homologous recombination. An RNAi selection cassette is suited for the production of targeting constructs since one copy of the cassette is sufficient for selecting an allele of the target gene which is modified by homologous recombination. If, in this case, the selection cassette is flanked by recognition sequences such as e.g. loxP, frt, attb-attp or the like for recombinases such as e.g. cre, flp, phiC31 or the like, it is possible to remove the selection cassette again in vivo by transfecting a recombinase expression vector. In case the HPRT construct is lost in a preferred embodiment and the cells express again HPRT, such clones can be enriched by means of HAT medium.

Thus, the invention further relates to a method for the production of a transgenic mammal comprising the following steps: (a) injection of the embryonic stem cell of the invention or of an embryonic stem cell selected according to the method of the invention into blastocysts of a mammal, (b) transfer of the blasotcysts into the uterus of a mammal, (c) carrying the transgenic mammal to full term. In a preferred embodiment, the transgenic mammal is a non-human transgenic mammal.

Furthermore, the invention relates to a method for the production of a transgenic mammal wherein the stem cells of the invention or the embryonic stem cells selected according to the method of the invention are (a) aggregated with blastomeres, (b) transferred into the uterus of a mammal and (c) the transgenic mammal is carried to full term. In a preferred embodiment, the transgenic mammal is a non-human transgenic mammal.

The technical teaching of the present invention can be used, in particular, to produce ES animals including ES mice. To this aim, the method, for example, of Schwenk et al. 2003 (Mol. Cell. Biol., 23:3982-3989) or of Nagy et al., 1990 (Development 110:815-821) can be applied. Here, diploid embryonic stem cells (ES), in particular the cells of the invention or cells which have been produced and/or selected according to the method of the invention are introduced into a tetraploid blastocyst which can be produced, for example, by means of electrofusion of fertilized egg cells at the 2-cell stage. The introduction of the cells of the invention can be carried out, for example, by microinjection; subsequently, the blastocysts are implanted into the uterus of surrogate mothers.

Furthermore, the invention relates to a method for the production of transgenic animals, wherein the nucleus of non-human cells of the invention is transferred into enucleated non-human oocytes.

In addition, the invention relates to a transgenic animal which contains a cell selected according to one or more step(s) of the method of the invention or which was produced according to the method of the invention for the production of a transgenic animal or for the production of a transgenic mammal. Preferred transgenic animals are mouse, rat, pig, bovine, primate, Danio rerio, C. elegans and Drosophila melanogaster. The transgenic animal is preferably a non-human transgenic animal.

At the same time, the invention comprises animals into which a cassette of the invention was introduced by means of microinjection into the male pronucleus of a fertilized egg cell.

According to the invention, it is possible to isolate pluripotent embryonic stem cells (ES cells) from the inner cell mass of blastocysts (embryos approximately on day 3, 5 of embryonic development). These cells are, for example, transfected in vitro with the construct of the invention. After a homologous recombination in the ES cells, the ES cells can be re-injected into blastocysts. The blastocysts are transferred into the reproductive tract of a surrogate mother and carried to full term.

In principle, viral as well as non-viral transfer systems can be used for gene transfer into the cells. Appropriate viral vectors comprise retrovirus, adenovirus, adeno-associated virus, Herpes virus, Vaccinia virus, Polio virus and the like. Alternatively, non-viral techniques can be applied for gene transfer, such as receptor-mediated, targeted DNA transfer using ligand DNA conjugates or adenovirus ligand DNA conjugates, lipofection, membrane fusion or direct microinjection. The production of transgenic animals is known to those skilled in the art and is carried out according to conventional methods (see e.g. Hogan, B., Beddington, R., Costantini, F. and Lacy, E. (1994), Manipulating the Mouse-Embryo; A Laboratory Manual, 2. Ed., Cold Spring Harbor Laboratory, Cold Spring Harbor, N.Y.).

Finally, the invention relates to a method for the production of a transgenic plant, comprising the steps: (a) culturing the protoplast of the invention or a protoplast produced according to the method of the invention in an appropriate growth medium and (b) regeneration of whole plants.

The plant cells of the invention can be used in the methods of the invention for the regeneration of whole, intact plants. Thus, the invention also relates to transgenic plants obtainable by regeneration of a plant cell of the invention as well as plants containing transgenic plant cells of the invention. Numerous methods and growth media for the culturing of plant cells and protoplasts, which can be used in the methods of the invention, are known to those skilled in the art. The selection of culture conditions depends on the cell concerned.

The Figures show:

FIG. 1a-d: HomoloGene database entries of the NBCI. The Figures a-d show the database entries of HomoloGene build 36 (last update May 25, 2004) of the following genes: HPRT HomoloGene: 162, APRT HomoloGene: 413, thymidine kinase HomoloGene: 2446, DHFR HomoloGene: 619.

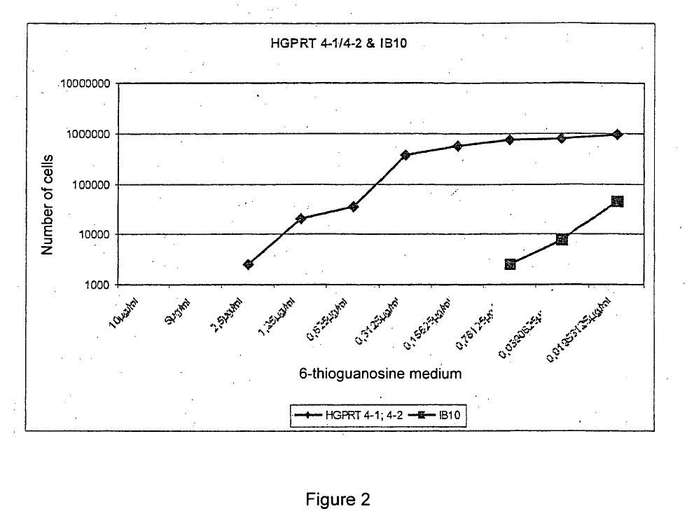

FIG. 2 Stably HPRT 4-1/4-2 transfected IB10 ES cells develop resistance against 6-thioguanosine. In order to examine the effect of the HPRT-RNAi construct 4-1/4-2, 10⁴ cells of a clone were seeded per well into a 24-well cell culture plate and cultured in ES medium with different concentrations of 6-thioguanosine for 8 days. Evaluation was carried out by determining the cell numbers after 8 days. As a control, non-transfected IB-10 ES cells were subjected to the same selection conditions.

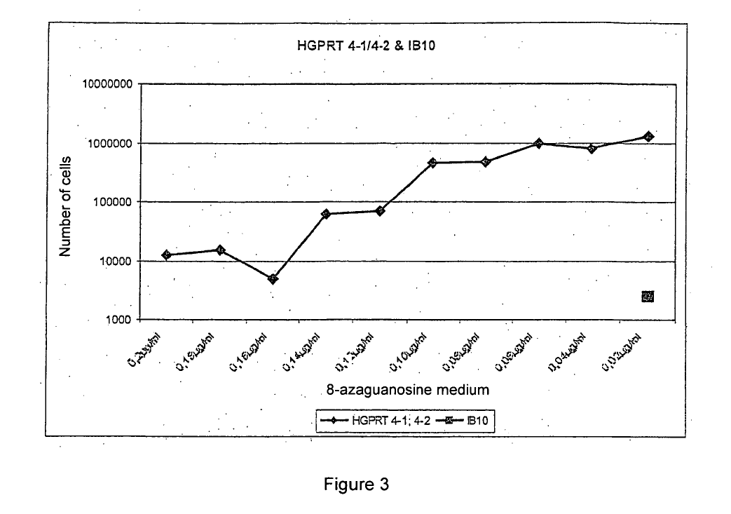

FIG. 3 Stably HPRT 4-1/4-2 transfected IB10 ES cells develop resistance against 8-thioguanosine. In order to examine the effect of the HPRT-RNAi construct 4-1/4-2, 10⁴ cells of a clone were seeded per well into a 24-well cell culture plate and cultured in ES medium with different concentrations of 8-azaguanosine for 8 days. Evaluation was carried out by determining cell numbers after 8 days. As a control, non-transfected IB-10 ES cells were subjected to the same selection conditions.

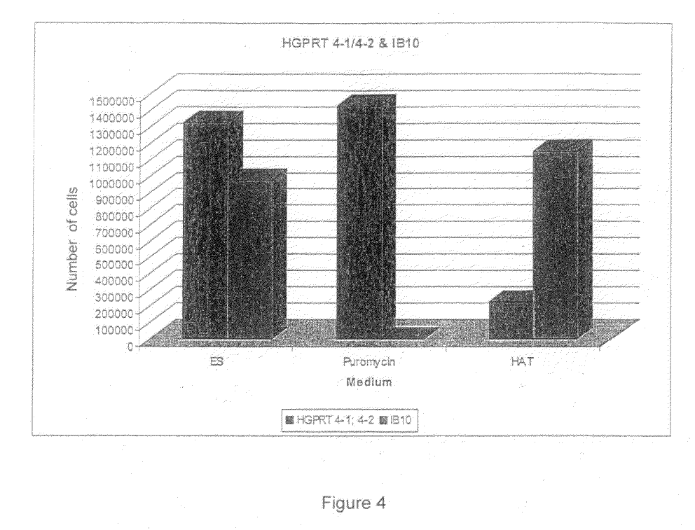

FIG. 4 Stably HPRT 4-1/4-2 transfected IB10 ES cells become sensitive to HAT medium. In order to examine the effect of the HPRT-RNAi construct 4-1/4-2, 10⁴ cells of a clone were seeded per well into a 24-well cell culture plate and cultured in ES medium without or with addition of puromycin or HAT for 8 days. Evaluation was carried out by determining cells numbers after 8 days. As a control, non-transfected IB-10 ES cells were subjected to the same selection conditions.

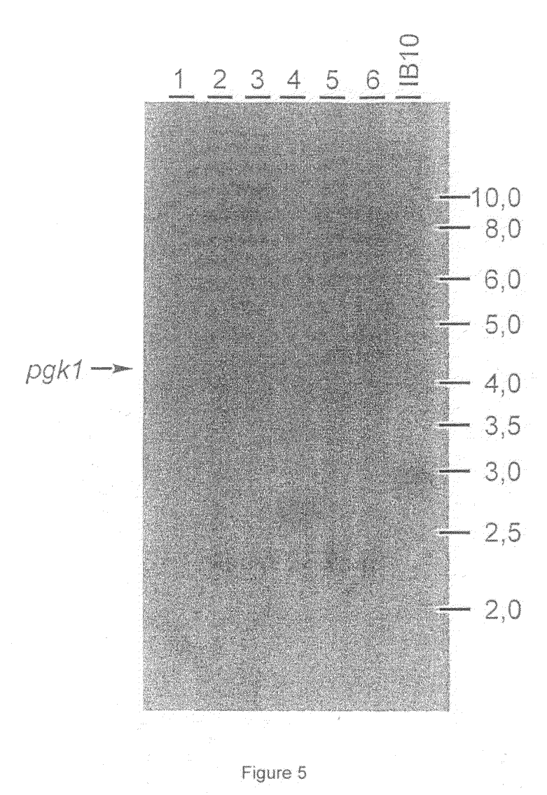

FIG. 5 Determination of HPRT-RNAi copy number in stably transfected IB10 ES cells: In order to determine the number of HPRT-RNAi copies, genomic DNA of IB10 ES cells clones stably transfected with construct 4-1/4-2 was digested with HindIII, separated by electrophoresis, blotted and hybridised with a 1040 bp long HindIII/SacII fragment of the HPRT-RNAi vector 4-1/4-2 (lines 1-6) which, apart from the H1 promoter and the HPRT sequences, also contains the pGK promoter of the puromycin restistance gene. Genomic DNA from non-transfected IB10 cells (IB10) were used as a control. pgk1 denotes the fragment which is produced by hybridisation with the endogenous pgk1 gene.

The Examples illustrate the invention:

### EXAMPLE 1

### Cloning of siRNA Constructs Directed Against Murine HPRT mRNA

According to the algorithm for synthetic double-stranded shRNA molecules developed by Tuschl and collaborators (Albashir et al., 2001), a sequence directed against murine HPRT-mRNA was identified.

Said sequence was synthesized as indirect repeat in a sense and anti-sense oligonucleotide (4-1 and 4-2), whereby both halves of the repeat were separated by a sequence section which forms a hairpin loop in the mRNA. At the ends, nucleotides were added on both strands for the generation of a BamHI overhang at the 5′ end and a HindIII overhang at the 3′ end. In order to generate double-stranded DNA fragments with the corresponding overhangs, the two single-stranded oligonucleotides were each mixed in an equimolar ratio, heated to 95° C. for 3 min and, subsequently, transferred into a 60° C. water bath which was slowly cooled to room temperature. Each of the two double-stranded DNA fragments generated in this way was subsequently ligated into the BglII/HindIII cleaved vector pSuper-retro and transformed in E. coli Dh5alpha. Plasmids that contained the fragment directed against HPRT were identified by restriction digestion of plasmid DNA from corresponding transformants. Identity of the constructs was verified by sequencing, the construct described below is referred to as 4-1/4-2.

### EXAMPLE 2

### Culture and Transfection of IB10 ES Cells

Murine embryonic stem cells were kept on gelatine-coated cell culture dishes without feeder cells under standard conditions (Torres and Kühn, 1997). For transfection, 10 μg of the transfecting HPRT-RNAi construct 4-1/4-2 were linearised using HindIII and then purified. 107 cells per experiment were transfected with 10 μg of plasmid DNA by means of electroporation. Subsequently, the cells were seeded on gelatine-coated cell culture dishes and kept in normal ES medium for two days. On the third day after transfection, the medium was replaced by selection medium (ES medium with 5 μg/ml puromycin) in which only stably transfected cells should survive. Developing clones were isolated after 8 days, further cultured individually, expanded and frozen.

### EXAMPLE 3

### HPRT-Dependent Selection

In order to examine the effect of the HPRT-RNAi construct, 104 cells of a clone were seeded per well into a 24-well cell culture plate and cultivated in different concentrations of HAT ES medium, ES medium with 6-thiguanosine and ES medium with 8-azaguanosine for 8 days. Cultures in ES medium without selection additives and ES medium with 5 μg/ml puromycin served as controls. In addition, non-transfected IB10 ES cells were subjected to the same selection conditions as control. Evaluation was carried out by determining the number of cells after 8 days.

After the transfection of HPRT-RNAi vectors in IB10 ES cells, puromycin resistant clones were obtained. The construct (4-1/4-2) described herein as an example developed resistance against 6-thioguanosine which is at least 10 times higher than the resistance of IB10 cells (FIG. 2). The same difference was observed with 8-azaguanosine (FIG. 3). At the same time, the cells were sensitive to HAT medium and their growth in this medium was six times worse than that of wild type cells (FIG. 4). In this case, the cells show the behaviour expected of IB10 without functional HPRT gene.

### EXAMPLE 4

### Determination of the Number of HPRT-RNAi Copies

In order to exclude that the effects observed with the selection are not based on the varying number of copies of HPRT-RNAi (construct 4-1/4-2) integrated into the genomes of the clones, the number of the integrated copies was determined by Southern Blot. For this purpose, genomic DNA isolated from the clones was digested with HindIII, separated on an agarose gel by electrophoresis and transferred onto a nylon membrane under alkaline conditions. After neutralisation, the DNA was hybridised with a 1040 bp long HindIII/SacII fragment of the HPRT-RNAi vector which, apart from the H1 promoter and the HPRT sequences, also contains the pGK promoter of the puromycin resistance gene. Due to this fact, the probe also hybridises with the endogenous pGK1 gene and the resulting band can be used for the quantification of the bands produced by the corresponding transgene.

The clones examined showed only one construct-specific band each which exhibited the same intensity as the band generated by the endogenous X-chromosomal pGK1 gene (FIG. 5). Due to the male genotype of the cell, IB10 have only one pGK1 copy. Consequently, the clones examined also carried only one copy of the HPRT-RNAi construct each. Apparently, one copy of the HPRT-RNAi construct is sufficient to inactivate the function of the endogenous HPRT gene to such a degree that it allows selection using 6-thiguanosine and 8-azaguanosine as well as negative selection using Hat medium.

1. A method for the production of a eukaryotic cell selectable by inactivation or reduction of an endogenous gene function, comprising the steps of
(a) introduction of one or more vectors into the cell and
(b) expression of a siRNA and preferably of shRNA coded by the one or more vectors, directed against an endogenous selectable gene and inactivating same, said siRNA or shRNA being the transcription product of an RNAi selection cassette, the selection cassette comprising a section of at least 19 nucleotides of the transcribed region of the gene, said section being operatively linked to a promoter and a transcription termination signal.

2. The method according to claim 1, comprising further step (c) of the expression of a recombinase, wherein the RNAi selection cassette is formed after homologous recombination, wherein before the recombination between the recombination precursors, between promoter and RNAi selection cassette or within the RNAi selection cassette a 5′ and a 3′ recombinase recognition sequence is located and between those a separating nucleotide sequence is located, wherein the separating nucleotide sequence contains a transcription termination signal and wherein a homologous recombination at the recombinase recognition sequences takes place as a consequence of the expression of the recombinase.

3. The method according to claim 2, wherein the recombinase recognition sequences are located between the first and the second sequence of the inverted sequence repeat of the shRNA.

4. The method according to claim 3, wherein
(i) the promoter, the 5′ recombinase recognition sequence and the first sequence of the inverted sequence repeat of the shRNA and
(ii) the 3′ recombinase recognition sequence and the second sequence of the inverted sequence repeat of the shRNA are located on different vectors.

5. The method according to any one of claims 1 to 4, comprising further step (d) of culturing and enrichment of cells containing and expressing the RNAi selection cassette, or (d′) culturing and enrichment of cells not containing and not expressing the RNAi selection cassette.

6. The method according to any one of claims 1 to 5, wherein the selectable gene is positively and/or negatively selectable.

7. The method according to any one of claims 1 to 6, wherein the endogenous selectable gene encodes a product capable of converting a non-selecting precursor of a substance A into a product B with selecting or selectable properties and wherein the cell is cultured in the presence of substance A.

8. The method according to any one of claims 1 to 7, wherein the non-selecting substance A is a non-toxic precursor of a toxin and product B is a toxin.

9. The method according to claim 7 or 8, wherein the gene is APRT, DHFR or TK.

10. The method according to claim 7 or 8, wherein the gene is HPRT.

11. The method according to claim 10, wherein 6-thioguanosine or 8-azaguanosine is used for selection.

12. The method according to any one of claims 1 to 11, wherein, together with the RNAi selection cassette or its recombination precursor, or subsequently, at least one additional nucleotide sequence is introduced into the cell.

13. The method according to claim 12, wherein the at least one additional nucleotide sequence are two nucleotide sequences with a homology to cellular genomic nucleotide sequences flanking the RNAi selection cassette or its recombination precursors at the 5′ and 3′ ends.

14. The method according to claim 12, wherein the additional nucleotide sequence introduced into the cell is located on the vector containing the RNAi selection cassette and the additional nucleotide sequence is enclosed by the 5′ and the 3′ flanking sequences with homology to cellular nucleotide sequences.

15. The method according to claim 12, wherein the additional nucleotide sequence introduced into the cell is located on a separate vector and wherein 5′ and 3′ of the additional nucleotide sequence, nucleotide sequences with a homology to cellular genomic nucleotide sequences are present.

16. The method according to claim 14 or 15, wherein the additional nucleotide sequence introduced into the cell is subsequently introduced into the cell and is flanked 5′ and 3′ by homologous genomic nucleotide sequences allowing for a recombination of the additional nucleotide sequence into the chromosomal position of the RNAi selection cassette.

17. The method according to any one of claims 12 to 16, wherein the RNAi selection cassette and/or the additional nucleotide sequence introduced into the cell is additionally flanked by recombinase recognition sequences.

18. The method according to any one of claims 12 to 17, wherein the additional nucleotide sequence introduced into the cell is a gene or a fragment thereof.

19. The method according to claim 19, wherein the gene is a mutant or an allelic variant of an endogenous gene of the cell.

20. The method according to claim 19, wherein the gene is an exogenous gene of the cell.

21. The method according to any one of claims 1 to 20, further comprising the following steps:
(e) inactivation or reduction of the RNAi expression; and
(f) selection and enrichment of cells not exhibiting an RNAi-based inactivation or reduction of the endogenous selectable gene.

22. The method according to claim 21, wherein the inactivation of the RNAi expression is a recombinase-mediated deletion and comprises the step of the expression of a recombinase.

23. The method according to claim 21 or 22, wherein the endogenous selectable gene is HPRT and the selection of cells not exhibiting an RNAi-based inactivation of the endogenous selectable gene takes place in the presence of HAT medium.

24. The method according to any one of claims 13 to 23, wherein the vector contains an additional cassette which upon random integration into a cellular gene leads to the inactivation thereof.

25. The method according to any one of claims 1 to 24, wherein the eukaryotic cell is a plant cell.

26. The method according to claim 25, wherein the plant cell is a protoplast.

27. The method according to any one of claims 1 to 24, wherein the eukaryotic cell is a vertebrate cell or an invertebrate cell.

28. The method according to claim 27, wherein the vertebrate cell is a mammalian cell.

29. The method according to claim 28, wherein the mammalian cell is a cell selected from human, mouse, rat, pig, bovine and primate.

30. The method according to claim 27, wherein the cell is an embryonic stem cell, an adult stem cell, a hematopoietic stem cell, a somatic cell or a cell of an established cell line.

31. Eukaryotic cell, comprising an RNAi selection cassette directed against an endogenous selectable gene and inactivating the function of said gene; wherein the RNAi selection cassette comprises a section of the gene with a length of at least 19 nucleotides which is operatively linked with a promoter and a transcription termination signal.

32. The cell according to claim 31, wherein the endogenous selectable gene is selected from the group consisting of HPRT, APRT, DHFR or TK.

33. The cell according to claim 31 or 32, wherein the RNAi selection cassette is stably integrated into the genome of the cell.

34. The cell according to any one of claims 31 to 33, wherein the cell is a plant cell.

35. The cell according to claim 34, wherein the plant cell is a protoplast.

36. Transgenic plant containing the plant cell according to claim 34 or 35.

37. The cell according to any one of claims 31 to 33, wherein the cell is a vertebrate cell or an invertebrate cell.

38. The cell according to claim 37, wherein the vertebrate cell is a mammalian cell.

39. The cell according to claim 38, wherein the mammalian cell is a cell selected from the group consisting of human, mouse, rat, pig, bovine and primate.

40. The cell according to claim 39, wherein the cell an embryonic stem cell, an adult stem cell, a hematopoietic stem cell, a somatic cell or an established cell line.

41. A method for the production of a transgenic animal, wherein
a) cells are treated according to one or more process steps mentioned in claims 1 to 30 and
b) a viable organism is grown from the cells.

42. A method for the production of a transgenic animal, comprising the steps of:
(a) injection of the embryonic stem cell of claim 40 or an embryonic stem cell selected according to the method of claim 30 in blastocysts of a mammal,
(b) transfer of the blastocysts into the uterus of a mammal, and
(c) carrying the transgenic mammal to full term.

43. Transgenic animal containing a cell selected according to one or more steps mentioned in claims 1 to 30, or containing a cell according to claims 31 to 33 or 37 to 40 or produced according to the method of claim 41 or 42.

44. A method for the production of a transgenic plant, comprising the steps:
(a) culturing the protoplast of claim 35 or a protoplast produced according to the method of claim 26 in appropriate growth medium, and
(b) regeneration of whole plants.
